(19) United States
(12) Patent Application Publication
(10) Pub. No.: US 2004/0186376 A1
(43) Pub. Date: Sep. 23, 2004

(54) Method and apparatus for improved surgical navigation employing electronic identification with automatically actuated flexible medical devices

(76) Inventors: Bevil J. Hogg, Santa Cruz, CA (US); Jeffrey M. Garibaldi, St. Louis, MO (US); Raju R. Viswanathan, St. Louis, MO (US)

Correspondence Address: HARNESS, DICKEY, & PIERCE, P.L.C, 7700 BONHOMME, STE 400, ST. LOUIS, MO, 63105, US

(21) Appl. No.: 10/674,914

(22) Filed: Sep. 30, 2003

Related U.S. Application Data
(60) Provisional application No. 60/414,574, filed on Sep. 30, 2002.

(51) Int. Cl. A61B 5/05

(52) U.S. Cl. 600/424; 600/117; 128/899

(57) ABSTRACT

A navigation system for minimally invasive surgery incorporating means of automatic electronic identification of automatically actuated flexible medical device characteristics, communication of such information to a navigation control system and its use in an automatic actuation system that is used to accurately control navigation of the flexible medical device within a patient's anatomy by the use of a physics-based model of flexible device response to automatically applied actuations in order to efficiently access specified regions targeted for therapy delivery.

### CROSS REFERENCE TO RELATED APPLICATION

This application claims priority of U.S. provisional patent application Serial No. 60/414,574, filed Sep. 30, 2002, the contents of which are incorporated by reference.

### BACKGROUND OF THE INVENTION

This invention relates to medical surgical navigation systems in which an automatically actuated elongate device is navigated to sites targeted within a subject's body, for example for therapy delivery, and in particular to the use of electronic identifiers with automatically actuated flexible medical devices for controlling the distal end of an elongate medical device for efficient surgical navigation and target access.

An increasing number of medical procedures are performed using elongate medical devices that are introduced into the body and navigated to the procedure site. While initially these medical devices were manually manipulated, automatic navigation systems have been developed to facilitate the navigation of medical devices through the body. These automatic systems include magnetic navigation systems, mechanical navigation systems, hydraulic navigation systems, and magnetostrictive and electrostrictive navigation systems. Many of these navigation systems have user interfaces and control systems that are device specific. It is important to insure that such navigation systems are compatible with the medical devices they control.

As a specific example, magnetic navigation systems have been developed which apply a controlled magnetic field to an operating region in a subject, to orient a magnetically responsive element on a medical device in the operating region. Examples of such systems include Ritter et al., U.S. Pat. No. 6,241,671, issued Jun. 5, 2001, for Open Field System For Magnetic Surgery (incorporated herein by reference). Magnetic navigation systems permit faster and easier navigation, and allow the devices to be made thinner and more flexible than mechanically navigated devices which must contain pull wires and other components for steering the device. Because of the advances made in magnetic navigation systems and magnetically responsive medical devices, the determination of the appropriate field direction, and instructing the magnetic surgery system to apply the determined magnetic field are probably the most difficult tasks remaining in magnetically assisted medical procedures. In co-pending, co-assigned, PCT Patent Application No. PCT/US03/24437, filed Aug. 1, 2003, which claims priority from U.S. patent application Ser. No. 10/448,273, filed May 29, 2003; U.S. Patent Application Serial No. 60/417,386, filed Oct. 9, 2002, and U.S. Patent Application Serial No. 60/401,670, filed Aug. 6, 2002, for Method and Apparatus for improved Magnetic Surgery Employing Virtual Device Interface (the disclosure of all of which are incorporated herein by reference) a virtual catheter interface has been described where a method for computation of an appropriate magnetic field required to steer the device in various ways has been described. The use of such an automatic computation system can be improved in several ways. One such significant improvement described herein is the incorporation of an electronic identifier with a magnetically steered flexible medical device for the purpose of efficiently navigating that specific device in a variety of situations within a patient.

Other uses of electronic identifiers with medical devices have been described in the past. In U.S. Pat. Nos. 6,266,551, and 6,427,314 (incorporated herein by reference), the use of an embedded chip for the purpose of identification of the device and calibration of its location sensor has been described. In this case the device provides electromagnetic location data and electrical mapping data to the system where the data is further processed and displayed in a variety of ways. In such an application, besides providing sensor calibration information the identifier may further serve to provide sufficient information to the system for the system to decide which software subsystems should be activated to utilize the appropriate kind of data processing corresponding to the device. Likewise, in Eto et al., U.S. Pat. No. 6,436,032 dated Aug. 20, 2002, (incorporated herein by reference) electronic identification is used in endoscopes to keep track of endoscope device usage and condition. Other electronic identifiers have been employed in the context of articulated mechanical robotic systems with graspers designed for surgery such as those described in Tierney et al., U.S. Pat. No. 6,331,181, dated Dec. 18, 2001, (incorporated herein by reference) where the device is electronically identified in order to enable an appropriate form of grasper control and kinematic articulation.

### SUMMARY OF THE INVENTION

In the field of minimally invasive interventional medicine, the use of systems that involve navigation of automatically actuated flexible medical devices within a patient's body, such as those employed in magnetic surgery, is an important new development that allows access to regions within a patient's body that are otherwise difficult to reach and demand a high level of skill from the physician. Correct device identification, including relevant physical characteristics that determine physical device response, is important for efficient navigation and control in the context of automatically actuated navigation systems. None of the prior art inventions discussed earlier addresses the issue of efficient navigational control within a subject of automatically actuated flexible devices used in such systems where flexible device physics must be employed. The present invention is designed to address this need.

The present invention relates to the management and control of elongate flexible medical devices used with automatically actuated navigation systems. In particular it provides a means of electronic identification of the device and its associated physical characteristics which enable efficient navigation through the use of appropriate navigation algorithms. The navigation system's user interface can employ the device information provided through this electronic identification to activate suitable navigation algorithms that enable device navigation as desired by the user. Thus according to one aspect of this invention, this management prevents the operation of the navigation system without a compatible medical device. This prevents the navigation system from being used with a device it was not designed to work with. According to another aspect of this invention, this management prevents medical devices from being reused, at least until they have been properly re-conditioned. This prevents the navigation system from being used with devices that may not be safe for the procedure. According to yet another aspect of this invention, the management and control system automatically configures itself for use with the elongate medical device connected it.

As an example, such a system might be used with a magnetic navigation system to control the specification and application of a magnetic field to the operating region in a patient to control the distal end of a medical device in the operating region. Generally the method of this invention comprises the use of a means of electronic identification of a flexible medical device used in an automatically actuated surgery system, the communication of the identification data to a navigation control system through a suitable interface, the selection of an appropriate set of navigation control software subsystems based on the device identification. This information, in combination with environmental information such as imaging data, electrical mapping data, temperature, etc., and user-specified desired target criteria for device navigation is used by the navigation system to compute corresponding actuation control, and apply the computed actuation control in order to navigate the device to the desired target(s).

In a preferred embodiment of the system and method of this invention, the medical device is constructed to be magnetically responsive by placement of suitable magnetically responsive materials on the distal portion of the device, and is steered by application of an external magnetic field. The external magnetic field may be applied by a variety of methods, including by the use of sets of external permanent magnets that are mechanically articulated so as to reorient the magnetic field in a specified navigation region and/or one or more electromagnets. The magnetically responsive flexible device is therefore automatically steered at its distal end by application of a suitable magnetic field. The device may be automatically advanced or retracted in the selected direction by using a device advancer system. The device is thus navigated through a combination of steering and advancement.

Efficient magnetic navigation requires that the proper field or sequence of fields is applied to the device so as to reach the desired target as directly as possible. This is best performed by employing an accurate model of the physics of the device's response, with user-specified targets or paths as inputs and corresponding magnetic fields to be applied as outputs. Such a model requires information about suitable physical and geometrical properties associated with the specific device that is being controlled.

In a preferred embodiment this information, together with a device type identification is stored electronically in a pod that is associated with (and preferably attached to) the proximal end of the device. In particular, the pod is connected to a navigation control system in this preferred embodiment whereby identification data including physical properties of the device can be relayed to the control system and a live connection (either by hard wire or by rf or other transmission) can be maintained between the device and the system to provide continuous information and to prevent substitution of the device. To ensure that the proper device characteristics are used in the navigation algorithms, the system can require a proper device identification for navigation and actuation to be enabled. The system is disabled for navigation purposes until such a proper device identification has been made. Furthermore, once an identification for a device has been made, that particular device can be enabled for navigation for a restricted period of time to ensure that the device is used in a single procedure only. Restriction to single use can be important because re-sterilizing the device or otherwise attempting to refurbish the device for re-use could unpredictably alter the physical characteristics of the device precluding tight control of the device in subsequent procedures. The type of device selected is also displayed on the system user interface upon identification providing an additional level of double checking. This allows a user to easily determine the type of medical device in use.

In an alternate preferred embodiment, the device is packaged together with a miniature radio frequency or RF transmitter that emits an electromagnetic signal, for example upon receipt of an interrogatory signal or upon the press of a button. This signal or “chirp” carries relevant device identification and other associated information. The control system is connected to a RF receiver that receives the electromagnetic signal and processes the information contained in the signal for use by the navigation control system. The control system will not enable device navigation until a proper identification has been made. In yet another alternate preferred embodiment, the device is paired with a “smart card” on which relevant device identification and associated information is stored, for example electrically, optically, magnetically, or in bar coded form, etc. “The smart card may or may not be packaged together with the device, it carries a packaging label with a tag that matches a tag on the device packaging, thus pairing each device with its own smart card. The navigation control system is connected to a smart card reader through which device identification and associated information may be read in from the smart card.” Again the control system will enable device navigation only when a proper identification has been made.

The pod or transmitter, or card may only contain device identifying indices which the navigation system uses to determine relevant physical properties in a look-up table. This allows for greater storage, and permits updating of the information without accessing each individual device.

The system and method of this invention allows the user to navigate a medical device efficiently by means of suitable control of a remote actuation system. This allows direct control of a medical device by remote means in a manner that allows optimal automated target access through the use of suitable physical and geometrical characteristics of the device in a physics model of the device's response. These and other features and advantages will be in part apparent, and in part pointed out hereinafter.

### BRIEF DESCRIPTION OF THE DRAWINGS

FIG. 1 is a schematic view of a surgery system incorporating the navigational control of an automatically actuated flexible interventional device provided with electronic identification of device type together with physical and geometrical properties as described in the present invention;

FIG. 2 is a schematic view of a preferred embodiment of a medical device designed to be automatically actuated by the application of a suitable external magnetic field and incorporating electronic identification in a pod mounted on the proximal end of the device;

FIG. 3A is a schematic view of an electronic identification pod construction;

FIG. 3B is an enlarged schematic view of the circuitry in the electronic identification pod showing the major electronic components that are involved in electronically identifying the device to the navigation control system.

Corresponding reference numerals indicate corresponding parts throughout the several views of the drawings.

### DETAILED DESCRIPTION OF THE INVENTION

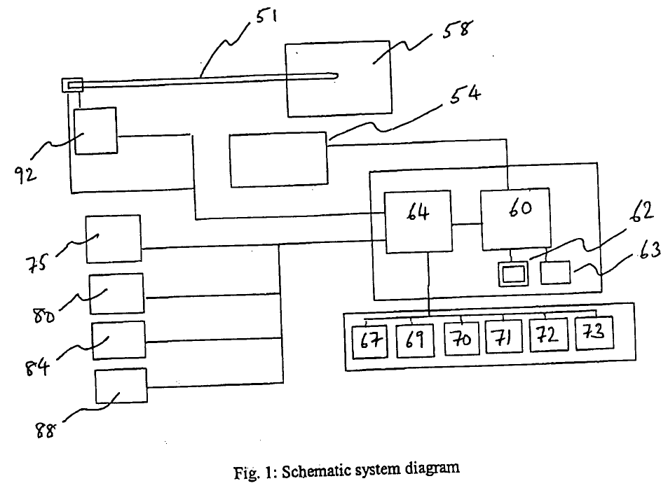

As shown in FIG. 1, a surgery system for automatic navigational control of an elongate flexible medical device 51 consists of an actuation system 54 that is controlled by a navigational control system 57. In a preferred embodiment, the actuation system 54 is a magnet system that creates a magnetic field that can be used to steer the device 51 within an operating volume 58 within a subject's body. In this case, a magnetically responsive element on the distal end of the medical device responds to the external magnetic field to change the direction of the distal end of the device. However, the navigation method could use any other method for changing the orientation of the distal end of the elongate medical device 51. For example, the navigation control system could operate conventional pull wires built into the elongate medical device, or it could hydraulically operate chambers built into the medical device, or operate magnetostrictive or electrostrictive elements built into the elongate medical device. The elongate medical device 51 could employ any navigational method for selectively changing the orientation of the distal end of the device.

The navigational control system 57 generally comprises an actuation controller 60 with a graphical user interface 62 and a set of input devices 63, and a workstation computer 64 that is connected to a graphical user interface 67 and other input devices such as a mouse 69, a keyboard 70, a pen tablet 71, a device advancer 72 and/or a joystick 73. An imaging system 75 is generally connected to the actuation controller 60 and the workstation computer 64. Other systems such as an ECG system 80, an ultrasound system 84, a device localization system 88 and a device distal tip temperature sensing system 92 may be connected to the workstation computer 64.

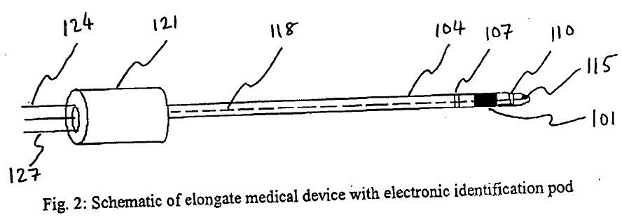

As shown in FIG. 2, in a preferred implementation the medical device 51 generally comprises a flexible and usually hollow shaft 97; a magnetically responsive element 101 that imparts device actuation in the distal portion 104 of the shaft; electrodes 107 and 110 on the distal portion of the shaft; and a thermistor, thermocouple or other temperature sensing device 115 in the distal portion. Electrical leads 118 extend back to the proximal end. A device identification pod 121 incorporating stored electronic identification information is affixed to the device at the proximal end, and power leads 124 and communication leads 127 connect the pod to the workstation computer. In alternate embodiments the magnetically responsive element 101 that imparts device actuation may be replaced by other types of actuation devices, such as mechanical, hydraulic, electrostrictive and magnetostrictive devices.

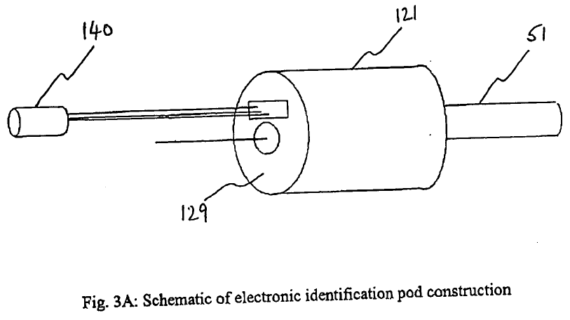

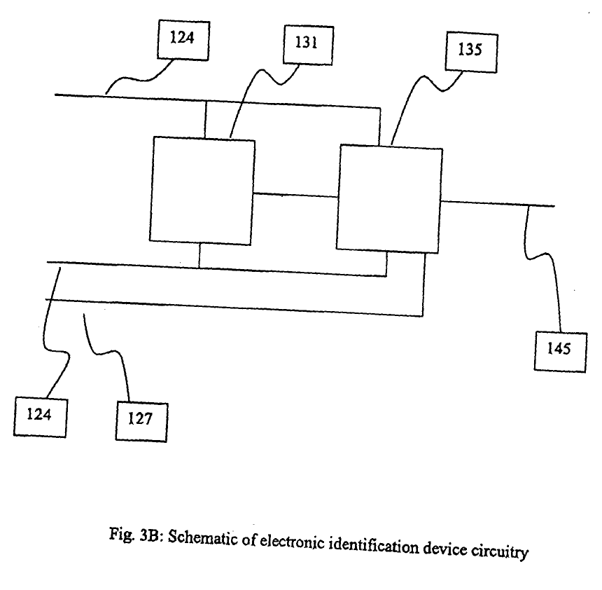

FIG. 3A shows the device identification pod 121 as consisting of electronic circuitry encased within an encasing 129 of protective material such as a polymer. Electrical leads 124 and 127 connect the pod 121 to the workstation computer 64. The leads 124 and 127 may be combined within a single connecting cable 140 that connects the pod 121 to the workstation computer 64. Of course the pod 121 does not have to be physically connected to the workstation computer, but can be linked by optical, rf or other means.

FIG. 3B is a schematic representation of the electronic circuitry within the device identification pod 121 which contains an electronic memory chip 131 such as an EPROM or other non-volatile memory storage chip for storage of device identification and device physical and geometrical properties, and an electronic chip 135 serving as a processing unit that handles communication with the workstation computer 64 through a communications link 127. Of course the pod 121 does not have to be physically connected to the workstation computer, but can be linked by optical, rf or other means. In a preferred embodiment, power for the electronics in the pod is derived from the workstation computer through the power leads 124. In a preferred embodiment the temperature sensing system consists of the temperature sensing device 115 connected to the processing unit 135 by means of leads 145, so that the sensed data is processed on the processing unit 135 before it is sent to the workstation computer 64.

In one implementation, when a medical device is plugged into the workstation computer through the leads provided from the pod, the workstation computer 64 recognizes the communications link and queries the pod 121 for identification. Upon receiving the query, device identification and associated device properties information is communicated to the workstation computer. For purposes of illustration, this characterization of device properties may include several quantities unique to the device that are essential for navigational control, such as lengths of flexible device segments, elastic properties of flexible device segments, device cross-sectional details, magnet dimensions, magnet type and other magnet characteristics. It may also include details about temperature dependence of the physical properties, specified by a set of functions {q(T)} where T is temperature.

In another implementation, a unique device identifier is communicated to the workstation computer 64 which then checks a database of identifiers to determine whether the device is a valid device. This database can also contain information about the type of device, or the physical and geometrical properties of the device, or this can be otherwise determinable from the device identifier.

In a preferred embodiment, if the identification is deemed to be valid, the workstation computer 64 starts to track the time of use of the medical device and enables the linking of software appropriate to the device 51 with the navigation control applications program. An invalid identification is so declared on the Graphical User Interface 67 and in this case navigation is not enabled. The identified type of medical device can be displayed on the workstation Graphical User Interface 67 for further verification. In a preferred embodiment, verification of device type is demanded on the Graphical User Interface 67 from the user as a further safety measure to ensure the correctness of device selection for the procedure about to be performed. In interventional medical procedures this can be an important matter since safety considerations often restrict the use of certain types of medical devices to certain procedures only, so that correct device identification is important. In a preferred embodiment, the device 51 is periodically queried for identification. In an alternate embodiment, the device 51 constantly sends status data including identification to the workstation computer 64 as long as it remains connected. Connectivity of the device may thus be monitored. If connectivity is lost, the device must be identified again. The total time of use of the device is tracked and restricted to be within a pre-defined limit as a safety measure to prevent re-use of the device under circumstances where its physical properties may have changed unpredictably as compared to the unused device rendering subsequent navigation control inaccurate. This time of use can be stored in the navigation system, at a central location accessible to all navigation systems, or even within the device itself. When a different type of medical device is plugged into the workstation computer, its identification allows the system to use an updated set of physical properties in its navigation control algorithms.

In an alternate version of the preferred embodiment, the circuitry in the device identification pod 121 is alterable, and its processing unit 135 carries a timer that keeps track of time elapsed since connectivity to the workstation computer 64 was established. After the medical device is used in a procedure or at the end of a pre-defined elapsed time, the processing unit 135 erases the device identification stored in the memory unit 131 so that a valid identification is no longer communicated to the workstation 64, rendering the navigation system and medical device inoperable. This prevents the elongate medical device incorporating the secure electronic identification from being improperly reused.

In one implementation, the navigation control system 57 automatically configures and adapts to the particular device, without the need for the user to program the system or take other action. In another implementation, the interface includes at least two computer programs that run on the processor, each adapted for a particular type of device. The information obtained from the memory allows the proper software for the device to run, and preferably prevents software adapted only for other devices from running.

The navigation control algorithm generally works as follows: The device identification stored in pod 121 discloses a set {p} of physical and geometrical properties (specified under certain standard conditions) relevant to the automatically actuated flexible device to the workstation computer 64. As stated earlier, this characterization may include one or more of several quantities such as lengths of device segments, elastic properties of the device segments, stiffness, device cross sectional details, magnet dimensions, magnet type and other magnet characteristics, the number of magnets and their spacing; and may also include details about temperature dependence of the physical properties, specified by a set of functions {q(T)} where T is temperature. In certain medical applications such as in electrophysiology, therapy delivery in the form of radio frequency (RF) ablation through electrodes such as 110 on the elongate medical device is employed, which serves to locally destroy unhealthy tissue within a patient's anatomy by raising the local temperature. In other cases, the distal end may include coils for creating a changeable magnetic moment at the tip, which can cause heating. Electrostrictive or other elements for shaping the distal end of the device may likewise cause heating. Temperature changes at the distal end of the device may in some cases affect the device's navigational characteristics and therefore it is useful to possess temperature dependence data exemplified by the set {q(T)}.

The workstation computer 64 also receives image information from the imaging system 75 and/or the ultrasound system 88, device localization information from the localization system 84, ECG information from the ECG system 80, and temperature information T from the temperature sensing system 92. Some or all of this information is processed by the workstation computer 64 to derive a set of variables {x} characterizing the current device distal tip configuration. Further, user inputs are accepted through any of the user input devices that may be the mouse 69, keyboard 70, pen tablet 71, device advancer 72 or joystick 73. These inputs generally dictate a choice of target location (parameterized by a set of variables {y}) that it is desired to access or a path or trajectory (parameterized by a set of variables {z}) that is desired for the device tip to follow. The navigation control algorithm determines a set of actuation control variables {u} which when applied drive the device towards the user-specified target criteria, given all the available inputs. Thus, the task of the navigation control algorithm is generally to specify or compute a functional relationship f such that

{u}=f({p}, {q(T)}, {x}, {y}, {z}) (1)

It is important to note that the functional relationship f is generally based on a physics model of flexible device response to applied actuations. In a preferred embodiment, the applied actuation consists of external magnetic fields and device advancements and retractions. An example of a physics model that determines the above functional relationship is detailed for example in co-pending, co-assigned. PCT Patent Application No. PCT/US03/24437, filed Aug. 1, 2003, which claims priority from U.S. patent application Ser. No. 10/448,273, filed May 29, 2003; U.S. Patent Application Serial No. 60/417,386, filed Oct. 9, 2002, and U.S. Patent Application Serial No. 60/401,670, filed Aug. 6, 2002, for Method and Apparatus for improved Magnetic Surgery Employing Virtual Device Interface (the disclosure of all of which are incorporated herein by reference).

In an alternate preferred embodiment, the electronic identification is placed on a miniature electronic device that is packaged together with the medical device or upon receiving a triggering signal. In this case, the electronic device is equipped with a miniature radio frequency or RF transmitter that emits a brief electromagnetic signal upon the pressing of a button on the device. This brief signal or “chirp” carries relevant device identification and other associated device properties information. An RF receiver that receives the electromagnetic signal and processes the information contained in the signal for use by the navigation control system is connected to the navigation control system. The control system will not enable device navigation until a proper identification has been made. In yet another alternate preferred embodiment, the device is paired with a “smart card” where relevant device identification and associated information is stored magnetically, by bar code or electronic, optical or other means. An example of a suitable smart card are 13.56 Mhz Secure RF Smart Card IC available from Amtel Corporation. Security is provided through the use of encrypted passwords, mutual authentication, data encryption and encrypted checksums.

A Contactless Smart Card system consists of an RF reader and an RF card. The reader emits an RF signal which polls for cards; data is exchanged when the card is within the RF field of the reader antenna. The RF card derives its power from the RF reader signal and does not require a battery or external power source.

To protect the fidelity of the information on the smart card, it may be expedient in some cases for it to be not packaged together with the device, but rather it may carry its own packaging label with a tag that matches a corresponding tag on the device packaging, thus pairing each device with its own smart card. The navigation control system is connected to a smart card reader where device identification and associated information may be read in from a smart card. Again the control system will enable device navigation only when a proper identification has been made.

While with these alternate embodiments of electronic identification of the automatically actuated medical device it may be expensive to enable and maintain a live connection between the device and the navigation control system, such embodiments without live connection may in some cases have the benefit of reduced cost. In such cases it may be more expedient to use these alternate means of electronic identification. In this situation if a different selection of medical device is made during the course of the procedure, the responsibility for ensuring that the new device is correctly identified (rather than the incorrect continued use of previously identified device properties) to the system is placed upon the system user or physician, who must enable the new device to be identified to the system by pressing an appropriate button on the packaged electronic device or reading into the system the smart card paired with the device. The preferred implementation uses an RF smart card (similar in appearance to a credit card without a magnetic stripe) packaged with the medical device that is read by an external card reader/writer connected to the system. The reader/writer constantly emits an RF signal, and when the RF smart card is brought in proximity to it, it reads data from the card (and also writes to the card, marking it as “already read” so it cannot be used again). This keeps the user workflow simple (the card can remain attached to the package while being read).

While the embodiments described herein are to be preferred, other means or principles of electronic device identification are conceivable to those familiar in the art and such principles are applicable according to the teachings of the present invention. Such embodiments may include among others electrical encoding with stored electrical charges, optical encoding similar to those employed in bar codes, or infrared or other electromagnetic transmission of identification information. Likewise, while a preferred method of remote actuation as described herein is based on magnetic actuation, the teachings herein also apply to other forms of remote actuation such as the use of magnetorestrictive materials, electrically controlled piezoelectric device actuation or other means of automatic actuation familiar to those skilled in the art of physics-based actuation.

What is claimed is:

1. A medical navigation system for controlling the distal end of an elongate flexible medical device in a subject's body, the system comprising:
an elongate flexible medical device together with an electronic identification device for elongate flexible medical device identification;
a navigation device for actuating the distal end of an elongate flexible medical device and thereby changing its orientation;
an electronic interface for selectively operating the navigation device for selectively controlling the orientation of the distal end of the elongate flexible medical device, the electronic interface comprising a processor and at least one software program that enables navigation control only in the presence of the electronic identification device.

2. The medical navigation system according to claim 1 wherein the electronic identification device includes a memory, and wherein the interface includes a reader for reading the memory.

3. The medical navigation system according to claim 1 wherein the electronic identification device includes a memory unit and a processing unit that communicates with the interface for transferring information.

4. The medical navigation system according to claim 2 wherein the memory contains unique identifying information about the type of device, and wherein the interface includes a database of the unique identifying information of the type of devices with which the interface is intended to operate.

5. The medical navigation system according to claim 3 wherein the memory contains unique identifying information about the type of device, and wherein the interface includes a database of the unique identifying information of the type of devices with which the interface is intended to operate.

6. The medical navigation system according to claim 1 wherein the electronic identification device is a circuit that is connected to the interface.

7. The medical navigation system according to claim 1 wherein the electronic identification device is a smart card with magnetically stored information that can be electronically read into the interface.

8. The medical navigation system according to claim 2 wherein the memory contains unique identifying information about the device, and wherein the interface includes a database of the unique identifying information for devices with which the interface is intended to operate.

9. The medical navigation system according to claim 3 wherein the memory contains unique identifying information about the device, and wherein the interface includes a database of the unique identifying information for devices with which the interface is intended to operate.

10. The medical navigation system according to claim 1 wherein the electronic identification device is a RF circuit that transmits a signal to the interface.

11. The medical navigation system according to claim 1 wherein the interface includes a plurality of programs, each adapted for use with a different type of elongate flexible medical device, each program operating only when an electronic identification device for the particular type of elongate flexible medical device is present.

12. The medical navigation system according to claim 1 wherein the electronic identification device includes an integrated circuit.

13. The medical navigation system according to claim 1 wherein the interface operates on the electronic identification device to prevent reuse of the elongate flexible medical device.

14. The medical navigation system according to claim 1 wherein the interface tracks elapsed time of use of the identified elongate flexible medical device and invalidates use of the identified elongate flexible medical device when the elapsed time exceeds a pre-defined limit.

15. The electronic identification device according to claim 3 wherein the processing unit operates on the memory unit to prevent reuse of the elongate flexible medical device.

16. The medical navigation system according to claim 1 wherein the electronic identification device includes memory, and wherein the interface adds to or deletes information stored on the memory to prevent reuse of the device.

17. The medical navigation system according to claim 1 wherein the at least one software program controls navigation by employing a computational model of flexible device physics.

18. A method of securing a medical navigation system from unauthorized use, the method comprising preventing the operation of at least one computer program of the medical navigation system except in the presence of an elongate flexible medical device having an electronic identification device.

19. The method of securing a medical navigation system from unauthorized use according to claim 18, further comprising altering the identification device after use of the medical device, to prevent reuse of the medical device.

20. A method of automatically adapting a medical navigation system for navigating the distal end of an elongate medical device, the method comprising reading a memory associated with the elongate device, and adapting the system based at least in part from the memory.

21. The method according to claim 20 wherein the step of adapting the system comprises running a program for the particular device as determined from the information read from the memory.

22. The method according to claim 20 wherein the step of adapting the system comprises using properties of the particular device as determined from the information read from the memory, in a program for navigation control.

23. A medical navigation system for navigating the distal end of an elongate flexible medical device inside a subject's body, the system comprising an elongate flexible medical device; a navigation device for actuating and orienting the distal end of the elongate medical device; an interface comprising a processor and at least one software program for selectively controlling the navigation device to selectively orient the distal end of the elongate medical device, the improvement comprising an electronic identification device provided with the elongate flexible medical device, which enables at least one navigation control software program of the interface to function.

24. The medical navigation system according to claim 23 wherein the at least one software program controls navigation by employing a computational model of flexible device physics.

25. The medical navigation system according to claim 23 wherein the electronic identification device includes a memory, and wherein the interface is adapted to read the memory.

26. The medical navigation system according to claim 23 wherein the electronic identification device is in the form of a smart card with magnetically stored information, and wherein the interface can access this information through the use of an electronic smart card reader.

27. The medical navigation system according to claim 23 wherein the electronic identification device is an integrated circuit including a memory, which is connected to the interface.

28. A medical navigation system for navigating the distal end of an elongate flexible medical device inside a subject's body, the system comprising an elongate flexible medical device; a navigation device for actuating and orienting the distal end of the elongate flexible medical device; an interface comprising a processor and at least two software programs each adapted for controlling the navigation device for a specific type of elongate flexible medical device to selectively orient the distal end of the elongate flexible medical device, the improvement comprising an electronic identification device provided with the elongate flexible medical device, which enables the appropriate navigation control software program for the particular medical device of the interface to function.

29. The medical navigation system according to claim 28 wherein at least one of the at least two software programs controls navigation by employing a computational model of flexible device physics.

30. The medical navigation system according to claim 28 wherein the electronic identification device includes a memory, and wherein the interface is adapted to read the memory.

31. The medical navigation system according to claim 28 wherein the electronic identification device is in the form of a smart card with magnetically stored information, and wherein the interface can access this information through the use of an electronic smart card reader.

32. The medical navigation system according to claim 28 wherein the electronic identification device is an integrated circuit including a memory, which is connected to the interface.

33. A medical navigation system for navigating the distal end of an elongate flexible medical device inside a subject's body, the system comprising an elongate flexible medical device; a navigation device for actuating and orienting the distal end of the elongate flexible medical device; an interface comprising a processor and at least one software program for selectively controlling the navigation device to selectively orient the distal end of the elongate flexible medical device, the improvement comprising an electronic identification device provided with the elongate flexible medical device, which provides information for the software program about the properties of the medical device.

34. The medical navigation system according to claim 33 wherein the at least one software program controls navigation by employing a computational model of flexible device physics.

35. The medical navigation system according to claim 33 wherein the electronic identification device includes a memory, and wherein the interface is adapted to read the memory.

36. The medical navigation system according to claim 33 wherein the electronic identification device is in the form of a smart card with magnetically stored information, and wherein the interface can access this information through the use of an electronic smart card reader

37. The medical navigation system according to claim 33 wherein the electronic identification device is an integrated circuit including a memory, which is connected to the interface.

38. A medical navigation system for controlling the distal end of an elongate medical device in the body of the patient;
an elongate flexible medical device;
a memory device provided with the flexible medical device;
a control system for controlling the position and/or orientation of the distal end of the elongate medical device;
an interface for accepting inputs from the user to cause the control system to selectively change the position and/or orientation of the elongate medical device; the interface sending instructions to the control system dependent in part upon information obtained from the memory device.

39. The medical navigation system according to claim 38 wherein the interface incorporates a software program that controls navigation by employing a computational model of flexible device physics.

40. The system according to claim 38 wherein the memory device includes storing unique device identification information for the elongate flexible medical device, and wherein the interface includes a database of unique device identification information and corresponding device properties, and wherein the instructions sent to the control system take into account the device properties determined from the database.

41. The system according to claim 38 wherein the memory device includes information identifying the type of device as well as device properties, and wherein the instructions sent to the control system take into account the device properties determined from the memory device.

42. A method of controlling an elongate flexible medical device inside a subject's body with a control system for controlling the position and/or orientation of the distal end of the elongate flexible medical device, the method comprising:
inputting information about the desired position and/or orientation to an interface which controls the control system based upon the input information and information about the properties of the device based on information read from a memory incorporated with the elongate flexible medical device.

43. The method according to claim 42 wherein the interface incorporates at least one software program that controls navigation by employing a computational model of flexible device physics.

44. A method of controlling an elongate flexible medical device inside a subject's body with a control system for controlling the position and/or orientation of the distal end of the elongate flexible medical device, the method comprising:
accepting inputs of information about the desired position and/or orientation to an interface;
determining information about the elongate flexible medical device from a memory provided with the device;
sending a control signal from the interface to the control system based upon the input information about the desired position and/or orientation of the distal end and the determined properties of the medical device.

45. The method according to claim 44 wherein the interface incorporates at least one software program that controls navigation by employing a computational model of flexible device physics.

46. The method according to claim 44 wherein the step of determining information about the elongate flexible medical device from a memory provided with the device comprises reading a unique device identifier from the memory, and determining the medical device properties from a database based upon the unique device identifier.

47. The method according to claim 44 wherein the step of determining information about the elongate flexible medical device from a memory provided with the device comprises reading a device type identifier from the memory, and determining the medical device properties from a database based upon the device type identifier.

48. The method according to claim 44 wherein the step of determining information about the elongate flexible medical device from a memory provided with the device comprises reading data corresponding to device properties from the memory.
